(19) United States
(12) Patent Application Publication
(10) Pub. No.: US 2025/0241585 A1
(43) Pub. Date: Jul. 31, 2025

(54) ENHANCING DIALOGUE INTERACTION SKILLS IN CHILDREN WITH AUTISM

(71) Applicant: Patrizia BONAVENTURA, Locust Valley, NY (US)

(72) Inventors: Patrizia BONAVENTURA, Locust Valley, NY (US); Krishnan PILLAIPAKKAMNATT, Westbury, NY (US)

(21) Appl. No.: 19/084,663

(22) Filed: Mar. 19, 2025

Related U.S. Application Data
(63) Continuation-in-part of application No. 18/886,665, filed on Sep. 16, 2024.
(60) Provisional application No. 63/538,326, filed on Sep. 14, 2023.

(51) Int. Cl. A61B 5/00; G09B 5/04; G16H 40/67

(52) U.S. Cl. CPC A61B 5/4803; G09B 5/04; G16H 40/67

(57) ABSTRACT

A computer-implemented therapy method for training conversation and dialogue interaction skills as well as pragmatic language skills in children with ASD is provided. The method includes: obtaining a robot having a non-humanoid, non-animal-like shape, the robot including at least one processor and memory coupled to the processor for controlling one or more actions of the robot; and configuring the processor to control the robot: (i) to monitor audible and visual expressions of a child with Autism Spectrum Disorder in proximity of the robot; and (ii) to audibly generate sentences pronounced by the robot in order to elicit a verbal response from the child and to trigger a conversation, the sentences being automatically generated as a function of the audible and visual expressions of the child to thereby initiate and maintain a conversation with the child.

### CROSS-REFERENCE TO RELATED APPLICATIONS

The present application is a continuation in part of U.S. application Ser. No. 18/886,665 filed Sep. 16, 2024, which claims priority to U.S. Provisional Application Ser. No. 63/538,326 filed Sep. 14, 2023, the contents of each of which are incorporated by reference in their respective entireties.

### FIELD

The present disclosure relates generally to the electrical, electronic and computer arts, and, more particularly, to techniques for enhancing dialogue interaction skills in children with autism.

### BACKGROUND

Children with autism spectrum disorder (ASD) frequently experience neurodevelopmental dysfunctions that affect their communication skills, social interactions, and ability to develop, maintain, and understand social relationships. (See, e.g., American Psychiatric Association, 2013, “Diagnostic and statistical manual of mental disorders: DSM-5,” 5th edition, Washington, DC: American Psychiatric Association (hereinafter “APA, 2013”), the disclosure of which is incorporated herein by reference in its entirety). Social communication deficits include impairments in aspects of joint attention and social reciprocity, as well as challenges in the use of verbal and nonverbal communicative behaviors for social interaction. Furthermore, children with ASD typically exhibit restricted, repetitive behavior apparent in stereotyped motor movements, insistence on or inflexible adherence to routines, abnormal intensity when fixated on interests, and hyper-or hypo-reactivity to sensory input (APA, 2013).

In terms of use of language and speech, children with ASD often experience difficulties in discourse and in conversation; in particular, they show a delay in, or total lack of, the development of spoken language (without compensation, for example, through gesture or mime). In individuals with adequate speech, a marked impairment is found in the ability to initiate or sustain a conversation with others (see, e.g., APA, 2013; So, et al., “Robot-based Play-drama Intervention May Improve the Narrative Abilities of Chinese-speaking Preschoolers with Autism Spectrum Disorder,” Res Dev Disabil., 2019 December; 95:103515, doi:10.1016/j.ridd.2019.103515. Epub 2019 Oct. 24. PMID: 31670026.; Loveland, McEvoy, Tunali, & Kelley, 1990, the disclosures of which are incorporated herein by reference in their entireties); in maintaining the dialogue by appropriate turn taking, and in selection of appropriate topics to continue the conversation, as well as in persevering on topics based on their own interest; in presence of stereotyped and repetitive use of language.

Children learn these unspoken rules of language interaction, during normal language development, starting soon after birth; research suggests they may be closely linked to play skill development. Some difficulties might be normal during acquisition of these rules, as in other areas of development, but if problems persist in the use of pragmatic language skills, and interfere with the child's normal conversations and language use with her/his peers and family, then it is possible that the child develops a pragmatic disorder, as manifested in autism.

Children with pragmatic language disorders have difficulties in verbal social interaction because they might not initiate conversation, may not take turns appropriately, may talk over another speaker, or respond with inappropriate silences. A child with pragmatic language delays may interrupt excessively, shift topics abruptly, or talk irrelevantly. They may assume that every listener has knowledge of the same people and events that they do. Children with pragmatic language delays may not be aware of the subtle cues people use to signal interest or discomfort. Their behavior may appear rude, distracted or self-involved. (Marasco et al., 2004).

Autism symptoms in children have been increasingly diagnosed, and the prevalence of autism in all regions of the world is high; 1 in 68 children is diagnosed with ASDs, according to the U.S. Centers for Disease Control and Prevention (CDC), occurring in all racial, ethnic, and socioeconomic groups. Furthermore, ASDs are almost five times more frequent among boys (1 in 42) than among girls (1 in 189) (CDC report, 2012).

From a linguistic point of view, children with autism show impaired pragmatic language skills and have difficulties in using speech and language in communication. Pragmatic language skills generally consist of knowing and using rules for normal verbal interaction with interlocutors; for example, establishing and maintaining eye contact during a conversation, smiling while talking, engaging others in a dialogue, maintaining comfortable speaking distances, taking conversational turns, changing topics, clarifying messages, and adding verbal or nonverbal information.

Prosody production deficits are one of the most common clinical features of Autism Spectrum Disorders (Edelson & Diehl, Edelson & Diehl, in press; McCann & Peppe, 2003; Paul, Augustyn, Klin, & Volkmar, 2005). Also, prosodic production differences are among the earliest characteristics of ASD (Oller et al., 2010; Schoen, Paul, & Chawarska, 2011; Werner, Dawson, & Osterling, 2000; Wetherby et al., 2004), and are present at all levels of ability, including Asperger syndrome (Shriberg et al., 2001) and high-functioning autism (Diehl, Watson, Bennetto, McDonough, & Gunlogson, 2009; Peppé, McCann, Gibbon, O'Hare, & Rutherford, 2007). However, these prosodic abnormalities, especially those in the production of intonation (the “melody of speech”), are not clearly defined and classified in their features, because this deficit has not been extensively studied (Diehl & Berkovits, 2010; McCann & Peppé, 2003; Peppé, 2009).

A diagnostic tool for prosody/intonation is not at present included in the ADOS-2 autism assessment test because, despite the fact that there is a general agreement on the presence of abnormal voice and intonation traits in these children, voice and prosody are not considered as a diagnostic indicator for ASD in the DSM-V and in the WHO definition of the disease (Shriberg, 2001; Fusaroli et al. 2017, Peppe' et al. 2003; WHO, 1993).

Therefore, there exists a need for a better characterization of these prosodic differences, especially in terms of intonation patterns, and to create assessment and therapy tools for prosodic disabilities (dysprosody) in children with ASD.

Finally, connected with the problem of the nature and causes of the dysprosody in verbal children with ASD, is the problem of the language spoken by the children and whether the deficits in pitch, loudness and intonation would be typical of each language, or common to all children with ASD across languages and cultures.

Data from production of prosody by children with ASD from different cultures, and speaking different languages, showed that prosodic patterns are indeed similar in these children at least for three languages (English, Italian and Tamil), indicating that the type of dysprosody might be a symptom of ASD independent from the prosody of the language the children with ASD speak.

Therefore, the robot was built to speak sentences in the three languages, Italian, English and Tamil, through a control App, and has been tested in different countries, providing good results in all the experimental conditions.

By using an App to control the robot's speech, also different models of piloted dialogues can be experimented, including different features in terms of semantic content (general topics and meanings of the sentences), of emotional content and of communication intentions (all these three levels of expression are related and conveyed by the intonation of speech): the manipulation of the intonation contours both in natural speech and in synthesized speech, can provide the necessary association between the appropriate response in dialogue interaction in terms of linguistic features (e.g. appropriate topic, adequate turns, comprehension of intentions of the speaker in the conversation) and the right prosodic features (like emphasis on the topic of the utterance and emotions expression in intonation).

The use of the robot together with different approaches of autism therapy (e.g. an ABA, or the DERBBI approach) would improve the use of language in interaction, and the ability to sustain a conversational exchange with appropriate linguistic pragmatic skills and intonation.

The children with ASD are known to have difficulty communicating with adults and peers, but they often demonstrate an affinity for technology, such as social robots. (See, e.g., Baron-Cohen, S., “The Hyper-systemizing, Assortative Mating Theory of Autism,” Progress in Neuro-Psychopharmacology and Biological Psychiatry, 30, 865-872, 2006). When interacting with social robots, children with ASD demonstrate prompted and spontaneous social behaviors, including joint attention, eye contact, understanding facial expressions and triadic interactions (See, e.g., Lee, H. et al., “The Intelligent Robot Contents for Children with Speech-Language Disorder,” 2015, Journal of Educational Technology & Society, vol. 18, no. 3, 2015, pp. 100-113; Johnson C. P., et al., “Identification and Evaluation of Children with Autism Spectrum Disorders,” Pediatrics 120(5), pp. 1183-1215, 2007; Kim, E. S., et al., “Social Robots as Embedded Reinforcers of Social Behavior in Children with Autism,” Journal of Autism Development Disorder, 43, pp. 1038-1049, 2013; K. H. Jeon, et al., “Robot-based Augmentative and Alternative Communication for Nonverbal Children with Communication Disorders,” In Proceedings of the 2014 ACM International Joint Conference on Pervasive and Ubiquitous Computing (UbiComp '14), Association for Computing Machinery, New York, NY, pp. 853-859, 2014, the disclosures of which are incorporated by reference herein in their entireties).

However, while social robots can be a powerful therapeutic tool for children with ASD who display an attraction towards technology and show “systemizing” skills, where they can recognize repeated patterns in stimuli (Duquette, Michaud and Mercier, 2008), no non-humanoid, non-animal-like robots are known to exist that are capable of eliciting or otherwise training conversation and dialogue interaction skills as well as pragmatic language skills in children with autism.

### SUMMARY

The present disclosure, as manifested in one or more embodiments, provides a unique robot-based therapy approach for training and improving conversation and dialogue interaction skills as well as pragmatic language skills in children with autism. In one or more embodiments, a non-humanoid non-animal-like robot is used as a behavioral therapy tool to speak with a child with ASD. In some embodiments, the robot is configured to move and approach the child, and to draw the child's attention by pronouncing some friendly questions.

The sentences pronounced by the robot aim to elicit a verbal response from the child, and to thereby start and maintain a conversation. In this manner, the robot is designed to improve verbal interaction in a dialogue and to enhance linguistic pragmatic skills used in interpersonal communication by spoken language in autistic children. Linguistic pragmatic skills comprise a body of knowledge relating to interactions in conversations; for example, knowing when to take turns, selecting an appropriate topic and staying on topic, or using an appropriate tone of voice in the dialogue, and sharing the correct expression of affect, among other conversational elements.

In accordance with one or more embodiments, a robot-and computer-implemented therapy method for training conversation and dialogue interaction skills as well as pragmatic language skills in children with ASD is provided. The method comprises: obtaining a robot having a non-humanoid, non-animal-like shape, the robot including at least one processor and memory coupled to the processor for controlling one or more actions of the robot; and configuring the processor to control the robot: (i) to monitor audible and visual expressions of a child in proximity of the robot; and (ii) to audibly generate sentences pronounced by the robot in order to elicit a verbal response from the child and to trigger a conversation, the sentences being automatically generated as a function of the audible and visual expressions of the child to thereby initiate and maintain a conversation with the child.

As may be used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed. For the avoidance of doubt, where an actor facilitates an action by other than performing the action, the action is nevertheless performed by some entity or combination of entities.

One or more embodiments of the disclosure or elements thereof can be implemented in the form of a computer program product including a non-transitory computer readable storage medium with computer usable program code for performing method steps for implementing at least a portion of the disclosure. Furthermore, one or more embodiments of the disclosure or elements thereof can be implemented in the form of a system (or apparatus) including a memory, and at least one processor that is coupled to the memory and operative to perform exemplary method steps. Yet further, in another aspect, one or more embodiments of the disclosure or elements thereof can be implemented in the form of means for carrying out one or more of the method steps described herein; the means can include (i) hardware module(s), (ii) software module(s) stored in a computer readable storage medium (or multiple such media) and implemented on a hardware processor, or (iii) a combination of (i) and (ii); any of (i)-(iii) implement the specific techniques set forth herein.

Techniques according to embodiments of the present disclosure can provide substantial beneficial therapeutic effects. By way of example only and without limitation, one or more embodiments of the disclosure provide techniques to train conversation and dialogue interaction skills as well as pragmatic language skills in children with ASD, the embodiments having one or more of the following advantages, among other important benefits:
provides a non-humanoid, non-animal-like robot configured for use in training and improving conversation and dialogue interaction skills as well as pragmatic language skills in children with ASD, as part of a unique therapy approach;
ability to engage in short directed conversations with children with autism, to thereby improve the child's ability to initiate and maintain dialogue with an interlocutor;
ability to trigger recognition in children with autism that there is an interlocutor present and trigger verbal interaction of the child with the conversation partner (e.g., eliciting appropriate content as a topic of the dialogue, and training appropriate pragmatic language skills to use in the conversation);
creates directed dialogue through a prescribed path of interaction;
uses real human voices so that behaviors learned during therapy might be transferred to actual human conversational interactions.

The present disclosure further provides a tool to record the dialogues interaction between the children with ASD and the robot, so allowing to analyze and to identify patterns of dysprosody in intonation in the children's speech, and to create better tools to be used to diagnose and treat this symptom of ASD.

Also, the intonation in the sentences spoken by the robot can be artificially adapted to the intonation used by the child, so allowing the robot to tune to the discourse level of the child, and to teach her/him how to produce the correct intonation in dialogue.

These and other features and advantages of the present disclosure will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIG. 1 is a diagram conceptually depicting robot classification for certain identified studies based on shape, ranked in order of most verbal responses elicited by the robot (top row) to least number of verbal responses (bottom row); and

FIGS. 2a, 2b and 2c depict at least a portion of an exemplary robot configurable for implementing a therapy method for training and improving conversation and dialogue interaction skills as well as pragmatic language skills in children with autism, according to one embodiment of the present disclosure.

### DETAILED DESCRIPTION

Principles of the present disclosure will be described herein in the context of an illustrative apparatus, system and therapy method for training and improving conversation and dialogue interaction skills as well as pragmatic language skills in children with autism (ASD). In one or more embodiments, a non-humanoid non-animal-like robot is used to speak with a child with ASD. Sentences pronounced by the robot aim to elicit a verbal response from the child, and to start a conversation. In this manner, the robot is designed to train and improve verbal interaction and to enhance linguistic pragmatic skills used in interpersonal communication by spoken language in autistic children. It is to be appreciated, however, that the disclosure is not limited to the specific apparatus, system and/or methods illustratively shown and described herein. Rather, it will become apparent to those skilled in the art given the teachings herein that numerous modifications can be made to the embodiments shown that are within the scope of the claimed disclosure. That is, no limitations with respect to the embodiments shown and described herein are intended or should be inferred.

Autistic children that have difficulty communicating with adults and peers often demonstrate an attraction for technology, such as social robots. However, while it is known to use social robots for assessing speech and language disorders in children (see, e.g., U.S. Pat. No. 11,145,217 to Chen et al., the disclosure of which is incorporated by reference herein in its entirety), there is presently no known therapy method or system that employs a non-humanoid non-animal-like robot for conversation and dialogue interaction skills as well as pragmatic language skills in children with autism ASD.

There have been various studies conducted using different types of robot shapes ranging from humanoid to non-humanoid form and their application to improvement of communication skills for children with ASD. Ample consideration has been given to robot appearance and function in ASD research. Some robots are humanoids, while others are mechanical and non-humanoid, and still others are animal-like Humanoid robots resemble humans, but remain repetitive and predictable in character. Using humanoid robots in interaction with children with ASD is beneficial because there may be a potential for generalization, meaning the child will use the behavior learned in research or clinical settings and transfer that learned behavior to her/his daily life (see, e.g., S. Shamsuddin, et al., “Humanoid Robot NAO Interacting with Autistic Children of Moderately Impaired Intelligence to Augment Communication Skills,” Procedia Engineering, 41, 2012, pp. 1533-1538, the disclosure of which is incorporated by reference herein in its entirety). For this reason, humanoid robots can aid in behavioral imitation exercises in targeted ASD therapy.

A drawback to using robots having a humanoid shape, however, is that they are less engaging to children with autism compared to non-humanoid robots. Children with ASD often withdraw themselves from human interactions and gravitate towards simple, repeatable, mechanical objects. In addition, many children with ASD can experience sensory overstimulation when exposed to an abundance of social cues: in humanoid robots, there can be a greater degree of confusing or distracting stimuli compared to other robot shapes.

Animal-like robots are modeled after animals and appear social without risking sensory overstimulation in children with ASD. These robots often are designed to train much more simplistic social cues compared to those of humanoid robots while being easier to interpret because they often allow for the expression of social cues that are simpler to decipher than those from humanoid robots (see, e.g., K. H. Jeon, et al., “Robot-based Augmentative and Alternative Communication for Nonverbal Children with Communication Disorders,” In Proceedings of the 2014 ACM International Joint Conference on Pervasive and Ubiquitous Computing (UbiComp '14), Association for Computing Machinery, New York, NY, 2014, pp. 853-859, the disclosure of which is incorporated by reference herein in its entirety). However, animal-like robots cannot simulate human-human interactions.

Non-humanoid non-animal-like robots do not resemble either human or animal form and character. Using non-humanoid non-animal-like robots in ASD research is beneficial because they can be built to complete specific tasks, are simplistic in design, and facilitate engaging interactions between child and robot. Children with ASD often withdraw themselves from human interactions; robots in a toy form often do not prompt this behavior, which can allow for greater engagement than with humanoid robots (see, e.g., A. Duquette, et al., “Exploring the Use of a Mobile Robot as an Imitation Agent with Children with Low-functioning Autism,” Autonomous Robots 24 (2), pp. 147-157, 2008, the disclosure of which is incorporated by reference herein in its entirety). Like that of animal-like robots, non-humanoid non-animal-like robots cannot copy human-human interactions.

The challenge in designing a robot for ASD therapy, therefore, is finding a design that engages the child without overstimulating them. To create captivating but visually simplistic robots, a cartoon-like style may be adopted in robot design, where oversized primary features, such as the eyes, may be emphasized. Robots with cartoon-like faces and machine-like bodies may provide simple, engaging stimuli for children with ASD (see, e.g., H. Lee, et al., “The Intelligent Robot Contents for Children with Speech-Language Disorder,” Journal of Educational Technology & Society, vol. 18, no. 3, 2015, pp. 100-113, the disclosure of which is incorporated by reference herein in its entirety).

Robot mobility also plays an important factor in child engagement in an ASD therapy setting and can be used as a tool to achieve varying levels of organic motion. A robot with a greater number of degrees of freedom (DOF) in the head can appear more human-like than a robot with a stationary head. All robots in ASD therapy research have some movement capability, but typically lack the ability to roll or walk around their environment. A mobile robot can provide more variety in robot-human interaction because there is a greater number of activities a robot and child can achieve together. However, robot mobility is another factor in robot design, that can also increase the cost of development.

Robot design does not just encompass its appearance, but the clinical applications greatly influence their cosmetic and technical design. For example, if a child is set to improve their eye contact in a prescribed therapy session, a robot may be configured to have larger eyes or a camera in their eyes to track eye movement and maximize child engagement. The targeted behavior in ASD robot-assisted therapy will be factors affecting the robot's appearance and mobility in order to achieve a specific therapeutic goal.

In one or more embodiments of the disclosure, the shape of the robot is non-humanoid or non-animal-like and adapted to capture the attention of a child having ASD and to facilitate the initiation of interaction. One objective is to improve and increase the quality and complexity of spoken language exchanges in conversation

In researching clinical studies involving the use of social robots for assessing behavior of children with ASD, participants' characteristics, study method, and study results were identified. Participant age, sex, diagnosis, and group size were reported. Robot name, study design, and skills tested of some studies are discussed in detail below. Three variables were coded in each study: (i) Does the robot have a speech function; (ii) Is there a verbal speech response to the robot in a piloted dialogue; and (iii) Was the study used for targeted speech therapy in children with ASD.

In a first study by Lee et al. (Lee, J., Takehashi, H., Nagai, C., Obinata, G., & Stefanov, D., “Which robot features can stimulate better responses from children with autism in robot-assisted therapy?” International Journal of Advanced Robotic Systems, 9, 72, 2012), six ASD participants were assessed ranging in age from 6-15 years, with five participants being ASD males and one participant being an ASD female. In this study, social skills were measured, including eye contact, response to verbal cues and understanding facial expression. The design characteristics of the robot, Ifbot, were as follows: 45 cm in height; one degree of freedom (DOF), moves on two wheels; mobile arms; speaker; facial expressions, including moving eyes, eyelids, neck; LED lights in the head and mouth.

In a second study by Shamsuddin et al. (Shamsuddin, S., Yussof, H., Ismail, L. I., Mohamed, S., Hanapiah, F. A., & Zahari, N. I. (2012). Humanoid robot NAO interacting with autistic children of moderately impaired intelligence to augment communication skills. Procedia Engineering, 41, pp. 1533-1538), which involved a humanoid robot, NOA, five ASD participants were assessed ranging in age from 6-13 years, with four participants being ASD males and one participant being an ASD female. In this study, Gilliam Autism Rating Scale-Second Edition (GARS-2) was measured; GARS-2 is a screening tool developed by Dr. James E. Gilliam to serve as a practical piece of early childhood developmental screenings for autism spectrum disorders for individuals between the ages of 3 and 22. The design characteristics of the robot NAO were as follows: 58 cm in height; 25 degrees of freedom; internal navigation device for stability; balanced by pressure sensors in each foot; four speakers; on-board speech recognition and analysis system; two high-definition CMOS cameras that enable forward vision; semi-autonomous.

A related study by So et al. (So W C, Cheng C H, Lam W Y, Wong T, Law W W, Huang Y, Ng K C, Tung H C, Wong W, “Robot-based play-drama intervention may improve the narrative abilities of Chinese-speaking preschoolers with autism spectrum disorder,” Res Dev Disabil. 2019 December; 95:103515) incorporating the same robot, NAO, involved 26 ASD participants ranging in age from 4-6 years, with 23 participants being ASD males, and 3 participants being ASD females. This study measured narrative and gesturing abilities of the participants.

In a third study by Kim et al. (Kim, E. S., Berkovits, L. D., Bernier, E. P., Leyzberg, D., Shic, F. Paul, R., & Scassellati, B. (2013), “Social Robots as Embedded Reinforcers of Social Behavior in Children with Autism,” Journal of Autism Development Disorder, 43, pp. 1038-1049), which involved an animal-like robot, Pleo, 24 ASD participants were assessed ranging in age from 4-12 years, with 21 participants being ASD males, and 3 participants being ASD females. This study measured the number of utterances of the participants. The design characteristics of the robot Pleo were as follows: 17.8 cm in height; 16 degrees of freedom; light detection and navigation via a camera-based vision system; color camera and temperature sensor; touch, ground, tilt and force sensors; two speakers; speech-like vocal recordings; remote controlled; Life OS software platform.

In a fourth study by Wainer et al. (Wainer, J., Dautenhahn, K., Robins, B., & Amirabdollahian, F. (2014), “A pilot study with a novel setup for collaborative play of the humanoid robot KASPAR with children with autism,” International Journal of Social Robotics, 6, pp. 45-65; and Wainer J., Robins, B., Amirabdollahian, F., & Dautenhahn, K. (2014), “Using the humanoid robot KASPAR to autonomously play triadic games and facilitate collaborative play among children with autism,” IEEE Transactions on Autonomous Mental Development, 6, pp. 183-199) involving a humanoid robot, Kaspar, six ASD participants were assessed ranging in age from 8-9 years, with five participants being ASD males, and one participant being an ASD female. This study measured social behaviors of the participants, including eye gaze, spoken words and triadic interactions. The design characteristics of the robot Kaspar were as follows: 15 SFR sensors (located in the head, neck, arms, legs, feet and chest); hands contain neodymium magnets to enable the robot to hold objects in its hands; silicone skin; wireless connectivity; image processing library for visual recognition; speakers; interactive Sense-Think-Act architecture for semi-autonomous features.

In a fifth study by Vanderborght et al. (Vanderborght B, Simut R, Saldien J, et al., “Using the social robot Probo as a social story telling agent for children with ASD,” Interaction Studies: Social Behaviour and Communication in Biological and Artificial Systems. 2012; 13(3):348-372) involving a cartoon-like robot, Probo, four ASD participants were assessed ranging in age from 4-9 years. This study measured speech imitation of the participants through storytelling. The design characteristics of the robot Probo were as follows: 20 degrees of freedom; expresses emotion through facial expression; foam and fabric body material; lip-synch module allows its lips to move in time with its voice; remote controlled via Wizard of Oz.

In a related study by Simut et al. (Simut RE, Vanderfaeillie J, Peca A, Van de Perre G, Vanderborght B, “Children with Autism Spectrum Disorders Make a Fruit Salad with Probo, the Social Robot: An Interaction Study,” J Autism Dev Disord. 2016 January; 46 (1):113-126) using the same Probo robot, 35 ASD participants were assessed ranging in age from 5-7 years. This study measured eye contact of the participants.

In a sixth study by Duquette et al. (Duquette A, Michaud F, Mercier H (2008), “Exploring the use of a mobile robot as an imitation agent with children with low-functioning autism,” Autonomous Robots 24 (2):147-157) involving a humanoid-like robot, Tito, four ASD participants were assessed ranging in age from 4-5 years, with three participants being ASD males, and one participant being an ASD female. This study measured imitation of body language, imitation of words, and social behaviors, including eye contact and joint attention, of the participants. The design characteristics of the robot Probo were as follows: 60 cm in height; red, yellow and blue colors with soft, washable clothes; uses wheels to move, but has legs and feet to appear humanoid in shape; can express any prerecorded message; contains microphone-camera device in one eye; controlled via wireless remote control or Wizard of Oz settings.

The robots in the above studies were used as tools to improve the communication skills in children with ASD. The research question is ‘which robot shape is best for this application?’ Therefore, robot shapes were ranked in decreasing order of eliciting the greatest number of verbal responses from a child with ASD to the least number of verbal responses to determine which robot shape could be best used as a mediator for speech therapy for children with ASD.

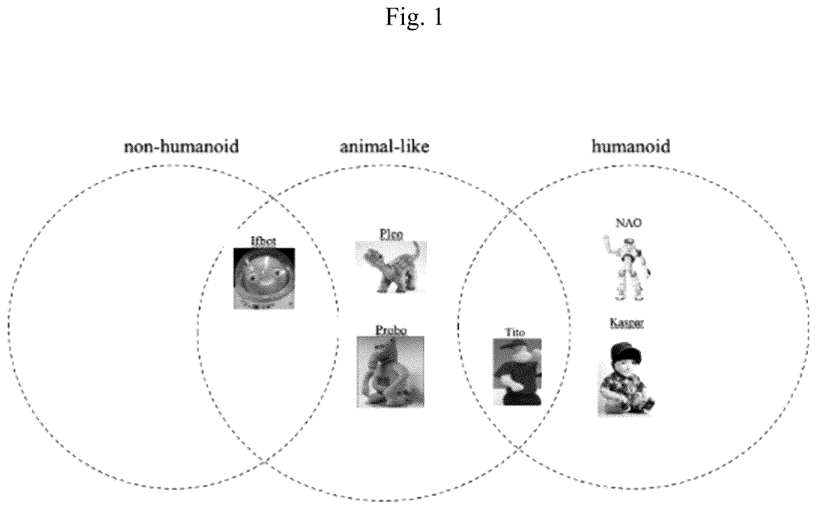

FIG. 1 conceptually depicts robot classification for the noted studies based on shape, ranked in order of most speech responses elicited by the robot (top row) to least number of responses (bottom row).

As a summary of results obtained from the noted studies, Shamsuddin et al. (2012) studied communication behaviors in five children with ASD between the ages of six and thirteen years in an interaction with the robot NAO. In this pilot study, NAO would move its body, blink, speak, and sing nursery rhymes, while experimenters measured child engagement and autistic behaviors using the GARS-2 test in a child-robot interaction.

Results indicate 80 percent of the ASD participants demonstrated improved communication and engagement in a robot interaction compared to in a typical classroom setting without a robot present. One child demonstrated less communication skills in a robot interaction. NAO had the capacity to reduce autistic behaviors on the communication subscale in a child-robot interaction for a majority of the study participants.

The So et al. (2019) study found that the robot NAO, while used in robot-based play-drama intervention, can improve narrative and gestural communication in children with ASD. A total of twenty six Cantonese-speaking children with ASD between the ages of four and six years participated in this study, with thirteen participants in the intervention group and the other thirteen participants in the control group. NAO would move, blink, and speak while acting out one of three narrative plays: “Farmer and Butterfly,” “Doctor and Patient,” or “Tourist and Tour Guide.” The children would then engage in a role-play activity with both the robot and human experimenter as experimenters measured the number of clauses, the proportion of complex clauses, the number of goal-based stories told, the number of inferences made, and the amount of gestures made by children with ASD. Results indicated children with ASD, when exposed to the robot assisted therapy condition, significantly increased the number of clauses, level of syntactic complexity, and amount of gestures compared to those not exposed to robot therapy. There was no significant change in affective inferences made after robot exposure. In both studies, NAO would speak with a synthetic voice in the primary language for the study participants.

Two studies have shown that NAO can improve the communication abilities in children with ASD that participate in robot assisted therapy (Simut et al., 2016; Wainer et al., 2014), so, for this reason, NAO was ranked as the humanoid robot that elicited the most speech responses from children with ASD.

Wainer et al. (2014 a) studied social behaviors in six children with ASD between the ages of eight and nine while playing an imitative, collaborative video game with each other and with the robot Kaspar. Like NAO, Kasper would move, blink, and speak while experimenters observed social behaviors in study participants, including displays of positive affect, or smiling and laughing, maintaining eye gaze, talking, and cooperating with the imitation game. Experimenters found Kaspar improves social behaviors, including displaying positive affect and maintaining eye gaze in all six children with ASD. However, there was no difference in communication behaviors where the children verbally responded or initiated conversation before and after robot interaction with one exception: the children did speak more while looking at another child playing with them when Kaspar was present than when Kaspar was absent. Because Kaspar was not found to significantly improve most communicative behaviors, Kaspar was ranked as the humanoid robot that elicited the least amount of speech responses from children with ASD.

Kim et al. (2013) found children with ASD produced more spontaneous utterances when interacting with the robot Pleo, a dinosaur-shaped robot that made animal sounds, than with an adult or touch screen computer game. The objective of the study was to determine the effectiveness of social robots as a reinforcer of social behavior, including verbal utterances, in twenty-four children with ASD between the ages of four and twelve years. The participants produced a mean 43 utterances in the presence of Pleo compared to a mean 36 utterances when interacting with an adult mediator. Pleo had the ages of five and seven years were tasked to build a fruit salad with Probo. The study found that only eye contact made by the capacity to improve verbal communication from children with ASD. Therefore, Pleo was ranked as the animal-like robot that elicited the most speech responses from children with ASD.

The robot Probo used social stories and imitation techniques to teach verbal cues to children with ASD (Wainer et al., 2014 b). In a pilot study, Vanderborght et al. (2012) analyzed the effect social stories in robot assisted therapy had on social responses of four children with ASD between the ages of four and nine. Each child had a different behavior targeted for therapy: the first had difficulty saying “thank you,” the second and third had difficulty sharing toys with other children, and the fourth had difficulty saying “hello.” The study found in social story intervention, Probo's presence decreased the level of prompting from a therapist needed to complete the social task. However, there was no significant difference in efficacy of storytelling therapy and storytelling therapy with Probo. Additionally, only one participant did not require prompting and engaged in their social task spontaneously. Verbal utterances, with the exception of two participants, were not measured in this study.

In another study, Probo was used to examine children's interaction with the robot compared to a human while playing a collaborative game (Simut et al., 2016). Here 35 children with ASD participated to the study. There was no change in social behaviors including joint attention, initiation, and displaying positive affect before and after robot interaction. There was also no significant difference in verbal utterances initiated before or after robot interaction. Measurement of verbal utterances was not the primary focus in the first study (Wainer et al., 2014 b) and the robot had no greater impact on verbal utterances made by children with ASD than a human partner. For these reasons, Probo was ranked as the animal-like robot that elicited the least amount of speech responses from children with ASD.

Ifbot is the only robot with a shape classified between non-humanoid and animal like shape which can elicit speech responses from children with ASD. Lee et al. (2012) studied social skills including eye contact, understanding facial expressions, and response to verbal cues in six children with ASD between the ages of six and fifteen years. They found children with ASD have a greater number of responses to verbal cues from the robot compared to with a human experimenter. Three typical cases were observed in this study. In Case 1, a female participant was quiet and reserved while interacting with a human prompter. When having a conversation with Ifbot, she remained quiet and turned her head the other way. Once Ifbot raised its arms, she answered some questions with yes/no. In Case 2, an active boy acted very interested and excited when interacting with Ifbot. He conversed with the robot until he was asked questions by the human prompter, in which he became distracted and the session ended. In Case 3, an active boy who typically talks to himself conversed with Ifbot once the robot started moving and speaking. After some time, the boy lost interest in Ifbot and started talking to himself again. The study participants were interested in Ifbot and responded more to verbal cues from the robot compared to a human prompter.

Duquette, Michaud, and Mercier (2008) studied the effect that the robot Tito had on facilitating reciprocal interactions, such as imitative play, in two pre-verbal and two non-verbal children with ASD between the ages of four and five years. The experimenters found pairing Tito with an autistic child had a positive impact on social behaviors, including imitation of facial expressions and reduced stereotyped behaviors, but a negative impact on word imitation. Examining communicative behaviors further, imitation of words mostly appeared for one preverbal participant, who repeated “bye bye” towards the end of the session. Although Tito was the only robot with speech features, having a shape between humanoid and animal-like, the study participants were not verbal and there was a negative impact on word imitation. Therefore, Tito was ranked as a robot that elicits less speech responses from children with ASD.

Generally, children with ASD display spontaneous verbal responses in the presence of a verbal robot. This behavioral observation is supported by some scientific evidence, but, due to the small number of participants in each experiment, the results cannot be considered representative of a more general effectiveness of the speaking robots.

Speech responses were elicited from children with ASD as they interacted with all robots mentioned in this review, indicating robots with both humanoid and animal-like shapes have the potential to be effective tools in improving communication skills in children with ASD.

When considering the robots NAO, Pleo, and Ifbot, there was an improvement in spontaneous verbal responses during or after exposure to a verbal robot. On the other hand, Kaspar, Probo, and Tito were ranked lower in eliciting verbal responses from children with ASD. As children with ASD interacted with these three robots, some social behaviors, like eye gaze and imitation, were improved. However, there was no significant change in speech productions in children with ASD while interacting with a human and with a robot. All six robots in the above-noted studies were classified as having non-humanoid or animal-like shapes, but only half were effective in eliciting spontaneous speech responses from children with ASD in their initial studies. The difference in communication behaviors displayed by children with ASD in each study could be explained by the amount of therapy a child has received and the level of communicative behaviors uniquely displayed by each individual.

In the above-noted studies, considering the spectrum of robots, from humanoid to non-humanoid in form, all had a speech function. However, no studies were found, highlighting the effectiveness of a verbal, non-humanoid non-animal-like robot used as a tool for targeted speech therapy in children with ASD. Furthermore, although it is known to use humanoid or animal-like robots for training motor, social and communication skills in children with autism, these robots focus only on acquisition of vocabulary and narrative skills.

In order to address at least the above-identified need in the field of therapy for children with ASD, one or more embodiments of the disclosure beneficially provides a behavior therapy methodology that incorporates a non-humanoid non-animal-like robot configured to train conversational and dialogue skills in children with autism. In this regard, aspects of the disclosure focus on improvement of both verbal interaction in dialogue and of linguistic pragmatic skills in use of language in speech interaction.

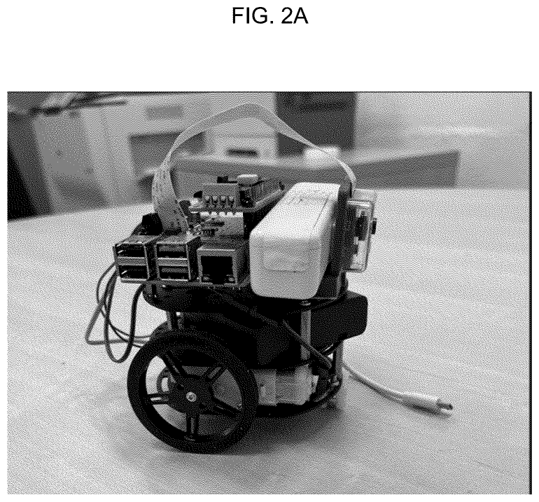

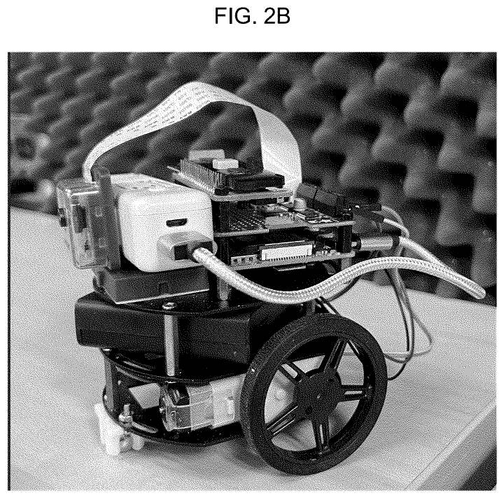

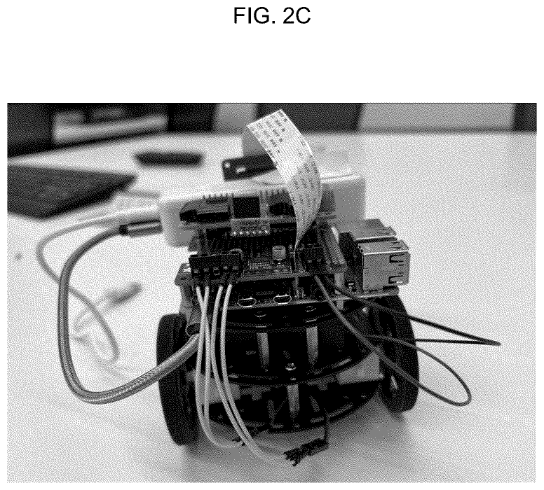

By way of example only and without limitation, FIGS. 2(a,b,c) depict at least a portion of an exemplary robot configurable for implementing a behavior therapy method associated with improving conversational dialogue in children with ASD, according to one or more embodiments of the disclosure. In one or more embodiments, the robot is a non-humanoid non-animal-like robot configured (i.e., programmed) to speak with a child with ASD. Sentences pronounced by the robot are designed to elicit a verbal response from the child, and to start and maintain a conversation. In one or more embodiments, the sentences pronounced by the robot are generated based on a human being in a natural voice (e.g., sampled human voice). The sentences may be pre-recorded and programmed in the robot to be elicited automatically by a command received from a remote control device (wired or wireless). In this manner, the robot seeks to improve verbal interaction in a dialogue and to enhance linguistic pragmatic skills used in interpersonal communication by spoken language in autistic children. Although one example of a robot is shown in FIGS. 2a, 2b, 2c, embodiments of the disclosure are not limited to any specific style, dimensions and/or features of the non-humanoid robot used in conjunction with the novel behavior therapy method for improving conversational dialogue in children with ASD.

With reference to FIGS. 2a, 2b, 2c, the exemplary robot comprises at least one processor, which in this embodiment is implemented using a Raspberry Pi 4 Model B computing module. The Raspberry Pi 4 module includes a processor, memory (e.g., random access memory (RAM)), and input/output (I/O) interface hardware (e.g., micro HDMI ports, gigabit Ethernet port, universal serial bus (USB) ports, etc.). The robot further includes a motor driver board adapted to control two or more DC steppers motors. The motors are attached to wheels for providing movement of the robot.

The robot may includes a sound module, which in one or more embodiments is implemented using a Raspiaudo sound card (part of the Raspberry Pi platform), includes two speakers and a microphone. The sound module is coupled to the Raspberry Pi computing module and provides an audio interface for the robot. A display module, which in one or more embodiments is preferably implemented using a MAX7219 serially interfaced 8-digit LED display, provides a visual interface for the robot for displaying expressions and other visual information generated by the computing module. The display module is coupled to the Raspberry Pi computing module using an interface module, which is implemented in one or more embodiments using an Adafruit Perma-Proto HAT board. A camera, which is coupled to the computing module, provides visual information to the processor, which can be used to monitor certain characteristics of the child, such as, for example, eye contact, facial expressions, etc., for evaluating prescribed social skills of the child.

A power module, which in this exemplary embodiment is implemented as a battery pack including four AA batteries, supplies power to the motor driver board and DC stepper motors. Additionally, a portable charger is provided to power the Raspberry Pi computing module, the Raspiaudo sound module, and the MAX7219 display module.

In one or more embodiments, the robot comprises computer program instructions which, when executed on the processor of the computing module, are configured to generate sentences designed to elicit conversational dialogue with the child. The dialogues of the children with the robot are recorded in audiovisual form by the robot, such as by using the on-board sound module and camera. Voice and speech acoustic parameters of the dialogue will be measured and analyzed to evaluate the progress of the child in engaging in conversational interaction with the robot.

More particularly, the quality of the verbal interaction in dialogue and the performance in pragmatic linguistic functions will be measured based on prescribed conversational parameters. Such prescribed conversational parameters may include, but are not limited to, one or more of the following: measuring the number of turns in the conversation; the number of appropriate turn taking; the number of initiations of dialogue; the number of topics selected by the child versus followed from the conversation; the number of appropriate topics selected by the child for the given conversation; the number of appropriate words used to express a topic; the number of different words/meanings selected; and the number of verbs and pronouns appropriately used overall and mean utterance length.

At least a portion of the apparatus of the present disclosure may be implemented in an integrated circuit. In forming integrated circuits, identical die are typically fabricated in a repeated pattern on a surface of a semiconductor wafer. Each die includes a device described herein (e.g., piezoelectric element, tuning inductor, etc.), and may include other structures and/or circuits. The individual die are cut or diced from the wafer, then packaged as an integrated circuit. One skilled in the art would know how to dice wafers and package die to produce integrated circuits. The exemplary apparatus illustrated in the accompanying figures, or portions thereof, may be part of an integrated circuit. Integrated circuits so manufactured are considered part of this disclosure.

Systems incorporating such integrated circuits are considered part of this disclosure. Given the teachings of the present disclosure provided herein, one of ordinary skill in the art will be able to contemplate other implementations and applications of embodiments of the disclosure.

The methodologies of embodiments of the present disclosure may be particularly well-suited for use in an electronic device or alternative system. Accordingly, embodiments of the present disclosure may take the form of an entirely hardware embodiment or an embodiment combining software and hardware aspects that may all generally be referred to herein as a “processor,” “circuit,” “module” or “system.” Furthermore, embodiments of the present disclosure, or portions thereof, may take the form of a computer program product embodied in one or more computer readable medium(s) having computer readable program code stored thereon.

Any combination of one or more computer-usable or computer-readable medium(s) May be utilized. The computer-usable or computer-readable medium may be a computer-readable storage medium. A computer-readable storage medium may be, for example but not limited to, an electronic, magnetic, optical, electromagnetic, infrared, or semiconductor system, apparatus, device, or any suitable combination of the foregoing. More specific examples (a non-exhaustive list) of the computer-readable storage medium would include the following: a portable computer diskette, a hard disk, a random access memory (RAM), a read-only memory (ROM), an erasable programmable read-only memory (EPROM or flash memory), an optical fiber, a portable compact disc read-only memory (CD-ROM), an optical storage device, a magnetic storage device, or any suitable combination of the foregoing. In the context of this disclosure, a computer-readable storage medium may be any tangible medium that can contain or store a program for use by or in connection with an instruction execution system, apparatus or device.

Computer program code for carrying out operations of embodiments of the present disclosure may be written in any combination of one or more programming languages, including an object oriented programming language such as Java, Smalltalk, C++ or the like, and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Embodiments of the present disclosure are described herein with reference to methods, apparatus (systems) and computer program products. It will be understood that any of the methods, apparatus and computer program products according to embodiments of the disclosure may be implemented using individual functional modules, blocks and/or circuits, and that combinations of such modules, blocks and/or circuits, may be implemented at least in part by computer program instructions running (i.e., executing) on one or more processing devices.

These computer program instructions may be stored in a non-transient computer-readable medium that can direct a computer or other programmable data processing apparatus or processor to function in a particular manner, such that the instructions stored in the computer-readable medium produce an article of manufacture including instruction means which implement a prescribed function/act according to one or more embodiments of the disclosure.

The illustrations of embodiments of the present disclosure described herein are intended to provide a general understanding of the various embodiments, and are not intended to serve as a complete description of all the elements and features of apparatus and systems that might make use of the circuits and techniques described herein. Many other embodiments will become apparent to those skilled in the art given the teachings herein; other embodiments are utilized and derived therefrom, such that structural and logical substitutions and changes can be made without departing from the scope of this disclosure. The figures are also merely representational and are not drawn to scale. Accordingly, the specification and figures are to be regarded in an illustrative rather than a restrictive sense.

Embodiments of the disclosure are referred to herein, individually and/or collectively, by the term “embodiment” merely for convenience and without intending to limit the scope of this application to any single embodiment or inventive concept if more than one is, in fact, shown. Thus, although specific embodiments have been illustrated and described herein, it should be understood that an arrangement achieving the same purpose can be substituted for the specific embodiment(s) shown; that is, this disclosure is intended to cover any and all adaptations or variations of various embodiments. Combinations of the above embodiments, and other embodiments not specifically described herein, will become apparent to those of skill in the art given the teachings herein.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Relational terms such as “upper,” “lower,” “front” and “back,” where used, are intended to indicate relative positioning of elements or structures to each other when such elements are oriented in a particular manner, as opposed to defining an absolute position of the elements.

The corresponding structures, materials, acts, and equivalents of all means or step-plus-function elements in the claims below are intended to include any structure, material, or act for performing the function in combination with other claimed elements as specifically claimed. The description of the various embodiments has been presented for purposes of illustration and description, but is not intended to be exhaustive or limited to the forms disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the various embodiments with various modifications as are suited to the particular use contemplated.

The abstract is provided to comply with 37 C.F.R. § 1.72(b), which requires an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the appended claims reflect, inventive subject matter lies in less than all features of a single embodiment. Thus the following claims are hereby incorporated into the Detailed Description, with each claim standing on its own as separately claimed subject matter.

All documents cited herein are incorporated by reference in their respective entirety. Given the teachings of embodiments of the disclosure provided herein, one of ordinary skill in the art will be able to contemplate other implementations and applications of the techniques of embodiments of the disclosure. Although illustrative embodiments of the disclosure have been described herein with reference to the accompanying drawings and figures, it is to be understood that embodiments of the disclosure are not limited to those precise embodiments, and that various other changes and modifications are made therein by one skilled in the art without departing from the scope of the disclosure, as manifested in the accompanying claims.

1. A computer-implemented therapy method for training and improving conversation, dialogue interaction skills, as well as pragmatic language skills and prosody in children with autism spectrum disorder (ASD), the method comprising:
obtaining a robot having a non-humanoid, non-animal-like shape, the robot including at least one processor and memory coupled to the processor for controlling one or more actions of the robot;
configuring the processor and a remote control device to control the robot: (i) to monitor and record audible and visual expressions of an autistic child in proximity of the robot; (ii) to audibly generate sentences pronounced by the robot in order to elicit a verbal response from the autistic child and to trigger a conversation, the sentences being generated to thereby initiate and maintain a conversation with the child; (iii) to record speech of other people in an environment through a remote control device.

2. The method according to claim 1 wherein the sentences pronounced by the robot are generated based on a human voice.

3. The method according to claim 1, wherein the sentences pronounced by the robot are pre-recorded and programmed in the remote control device that controls the robot, and are elicited automatically by a command received from the remote control device.

4. The method according to claim 3, wherein the remote control device is a wireless remote control device.

5. The method according to claim 3, wherein the remote control device is a wired remote control device.

6. The method according to claim 1, wherein the sentences pronounced by the robot are generated to initiate and continue a piloted dialogue with the autistic child.

7. The method according to claim 1, wherein the sentences pronounced by the robot are generated through a remote control device by a person controlling the robot.

8. The method according to claim 1, wherein the sentences pronounced by the robot are automatically generated by the robot, as directed by a user using a remote control device, to thereby start and maintain the conversation with the autistic child.

9. An apparatus for implementing a therapy method for training conversation and dialogue interaction skills as well as pragmatic language skills in children with autism spectrum disorder (ASD), the apparatus comprising:
a robot having a non-humanoid, non-animal-like shape, the robot including at least one processor and memory coupled to the processor for controlling one or more actions of the robot, the processor being configured to control the robot: (i) to monitor and record audible and visual expressions of an autistic child in proximity of the robot; (ii) to audibly generate sentences pronounced by the robot in order to elicit a verbal response from the autistic child and to trigger a conversation, the sentences being generated to thereby initiate and maintain a conversation with the autistic child; (iii) to record speech of other people in an environment through a remote control device.

10. The apparatus according to claim 9, wherein the processor is configured to generate the sentences pronounced by the robot as controlled by a person who operates the remote control device.

11. The apparatus according to claim 9, further comprising a remote control device in communication with the processor, wherein the sentences pronounced by the robot are automatically generated by the robot, as directed by a user using the remote control device, to thereby start and maintain the conversation with the autistic child.

12. The apparatus according to claim 9, further comprising a remote control device in communication with the processor, wherein the sentences pronounced by the robot are pre-recorded and programmed in the remote control device that controls the robot, and are elicited automatically by a command received from a remote control device.

13. The apparatus according to claim 12, wherein the remote control device is a wireless remote control device.

14. The apparatus according to claim 12, wherein the remote control device is a wired remote control device.

15. The method according to claim 1, further comprising allowing the robot to pronounce recorded speech of the other people in the environment as recorded through the remote control device, as if the robot were speaking the recorded speech to the autistic child.

16. The method of claim 7, further comprising the person controlling the robot recording speech by people in an environment, and playing the speech back to the autistic child through the robot, so appearing as if the robot was speaking the recorded speech to the autistic child.

17. The apparatus according to claim 9, where the apparatus is further configured to allow the robot to pronounce recorded speech of the other people in the environment as recorded through the remote control device, as if the robot were speaking the recorded speech to the autistic child.

18. The method of claim 1, where the environment is a room.

19. The apparatus of claim 9, where the environment is a room.

20. The method of claim 1, where the child is a patient undergoing treatment for autism spectrum disorder (ASD).
